(19) United States
(12) Patent Application Publication
(10) Pub. No.: US 2010/0179221 A1
(43) Pub. Date: Jul. 15, 2010

(54) FLUORINE-SUBSTITUTED AMPHETAMINES AND AMPHETAMINE DERIVATIVES AND USE THEREOF

(75) Inventors: Ulrich Nagel, Tubingen (DE); Werner Jürgen Schmidt, Tubingen (DE)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: Universitat Tubingen, Tubingen (DE)

(21) Appl. No.: 12/531,505

(22) PCT Filed: Mar. 19, 2008
(86) PCT No.: PCT/EP2008/002188

§ 371 Date: Dec. 22, 2009

(30) Foreign Application Priority Data
Mar. 19, 2007 (DE) 10 2007 014 286.4

(51) Int. Cl. A61K 31/197; C07C 211/03; A61K 31/137; A61P 25/28

(52) U.S. Cl. 514/567; 564/442; 514/646

(57) ABSTRACT

A fluorine-substituted amphetamine or amphetamine derivative with the formula (I):

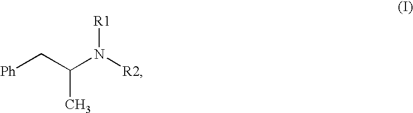

where at least one of the residues R1 or R2 is different from H and Ph is a phenyl ring, which is substituted with fluorine in at least one position or the residues R1 and R2 independently of one another are H or are different from H and Ph is a phenyl ring, which is substituted with fluorine in at least three positions or the residues R1 and R2 independently of one another are H or are different from H and Ph is a phenyl ring, which is substituted with fluorine in at least one position and has a substituent different from H in at least one other position.

### RELATED APPLICATIONS

This is a §371 of International Application No. PCT/EP2008/002188, with an international filing date of Mar. 19, 2008 (WO 2008/113565 A1, published Sep. 25, 2008), which is based on German Patent Application No. 10 2007 014 286.4, filed Mar. 19, 2007.

### TECHNICAL FIELD

This disclosure relates to fluorine-substituted amphetamines and amphetamine derivatives, use thereof as active substance and pharmaceutical compositions that comprise at least one fluorine-substituted amphetamine or amphetamine derivative.

### BACKGROUND

Neurological diseases, such as Parkinson's disease, can, starting from the brain, have effects on many parts of the human body. The four main symptoms of Parkinson's disease are rigor (muscle stiffness), tremor (muscle trembling) and hypokinesia (diminished movement), which can lead to akinesia (absence of movement), and postural instability. In addition there are cognitive deficits, principally deficits in implicit learning.

Treatment of neurological diseases and of their consequences often proves extremely complicated from the medical standpoint, despite modern methods of diagnosis and therapy. Often the causes of neurological disturbances are inadequately known and there is a lack of effective potential treatments.

For the patients affected, treatment of a neurological disease as a rule involves considerable upheavals in their life. In the case of treatment with L-DOPA, by far the best known antiparkinsonism medication, a protein-reduced diet is required, to ensure absorption of L-DOPA in the intestine, and discipline in taking the medication. Long-term observations of the course of the disturbances caused by the disease are necessary. Regularly recurring checks must be carried out, to ensure a sufficient supply of medication for the patient, in particular, adequate adjustment of the medication. For those affected this means increased need for advance planning of one's life, connected with considerable impairment of quality of life.

As a rule, with the drugs known at present, there can only be symptomatic treatment of neurological diseases. The underlying degeneration of dopaminergic neurons can as yet not be slowed down. Furthermore, long-term treatments often lead, as a side effect, to damage and/or impairment of other bodily functions. Thus, longer-term treatment with the aforementioned L-DOPA often causes dyskinesias, which until now can only be treated inadequately, if at all. In some cases, in long-term treatment with drugs habituation can occur, which, viewed over a longer period, necessitates a higher dosage of the drug. For the above reasons a change of medication may be required over the course of the treatment.

Accordingly, there is still a considerable need for improved active substances and drugs for the treatment of neurological diseases and their sequelae.

The work by Schmidt et al. (W. J. Schmidt, A. Mayerhofer, A. Meyer, K. Kovar, “Ecstasy counteracts catalepsy in rats, an antiparkinsonian effect?”, Neuroscience Letters 330 (2002) 251-254) describes antiparkinsonian activity of amphetamine derivatives, in particular, of 3,4-methylene dioxymethamphetamine (abbreviated to MDMA, also known as “Ecstasy”). The efficacy of MDMA was tested on rats. However, as is well known, MDMA has a strongly hallucinogenic potential, which basically opposes therapeutic use. Furthermore, toxic intermediates can form during degradation of MDMA in the human body.

It could therefore be helpful to provide active substances and medicinal products that permit improved treatment of neurological diseases and their sequelae, but preferably are less toxic than known active substances.

### SUMMARY

We provide a fluorine-substituted amphetamine or amphetamine derivative with the Formula (I):

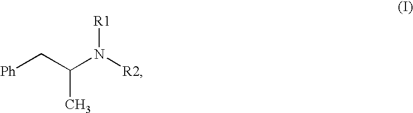

where a) at least one of the residues R1 or R2 is different from H and Ph is a phenyl ring, which is substituted with fluorine in at least one position or b) the residues R1 and R2 independently of one another are H or are different from H and Ph is a phenyl ring, which is substituted with fluorine in at least three positions or c) the residues R1 and R2 independently of one another are H or are different from H and Ph is a phenyl ring, which is substituted with fluorine in at least one position and has a substituent different from H in at least one other position.

We also provide a method for treating neurological diseases, their sequelae and/or treating side effects of a therapy of neurological diseases including administering a therapeutically effective amount of a fluorine-substituted amphetamine or amphetamine derivative to a patient.

We further provide a pharmaceutical composition including at least one fluorine-substituted amphetamine or amphetamine derivative as active substance.

### BRIEF DESCRIPTION OF THE DRAWINGS

FIG. 1 is a graph showing results of descent tests.

FIG. 2 is a graph showing results of dyskinesia tests.

### DETAILED DESCRIPTION

An amphetamine is to be understood as a chemical compound with the structure

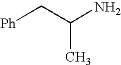

in which Ph stands for a phenyl ring. An amphetamine derivative is to be understood as a compound with the above structure, which has at least one residue different from H on the nitrogen and/or at least one substituent different from hydrogen (apart from fluorine) on the phenyl ring.

A fluorine-substituted amphetamine or amphetamine derivative has Formula (I):

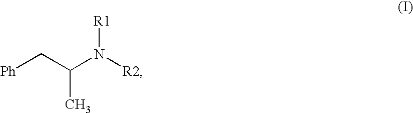

where
at least one of the residues R1 or R2 is different from H and Ph is a phenyl ring, which is substituted with fluorine in at least one position

or
the residues R1 and R2 independently of one another are H or are different from H and Ph is a phenyl ring, which is substituted with fluorine in at least three positions

or
the residues R1 and R2 independently of one another are H or are different from H and Ph is a phenyl ring, which is substituted with fluorine in at least one position and has a substituent different from H in at least one other position.

We showed in animal experiments that fluorine-substituted amphetamines or amphetamine derivatives are eminently suitable in particular for the treatment of neurological diseases and/or their sequelae and for the treatment of side effects of a therapy of neurological diseases.

Surprisingly, in addition, it was found that the amphetamine or amphetamine derivative in the body of an animal, in contrast to MDMA, is at most subject to very slight degradation or metabolization. This might be attributable to the fact that the fluorine-substituted phenyl ring of the amphetamine or amphetamine derivative, relative to the oxysubstituted phenyl ring of MDMA, gives rise to increased stability against metabolization of the amphetamine or amphetamine derivative.

This has the advantage that formation of toxic, in particular, neurotoxic metabolites or toxic, in particular, neurotoxic reaction products as a result of treatment with the fluorine-substituted amphetamine or amphetamine derivative essentially does not occur or only occurs to a slight extent. Preferably, the fluorine-substituted amphetamine or amphetamine derivative is excreted unchanged during or after the treatment.

Furthermore, the fluorine-substituted amphetamine or amphetamine derivative preferably has a so-called “sustained” action, i.e., it remains in the body of an animal or a human longer. In contrast to conventional drugs with so-called “pulsatile” profile of active substance, by using the fluorine-substituted amphetamine or amphetamine derivative it is possible to avoid a sudden high concentration of active substances (active substance spikes). Therefore, uniform supply of the active substance to a patient can be ensured. In comparison with active substances that are metabolized, the fluorine-substituted amphetamine or amphetamine derivative can be used in a relatively small dose in a therapy or a treatment.

The fluorine-substituted amphetamine or amphetamine derivative can in particular also be in the form of a salt, especially preferably in physiologically compatible form. The salt can in particular be a water-soluble salt, for example, a hydrochloride, sulfate or nitrate.

As a rule the fluorine-substituted amphetamine or amphetamine derivative is in the form of a mixture of enantiomers, i.e., as a racemate. The enantiomers can basically be present in the racemate in any relative proportions. It may be preferable for one of the enantiomers to be present in excess. Furthermore, it may be preferable for the fluorine-substituted amphetamine or amphetamine derivative to be in enantiomerically pure form.

The residues different from H, which the amphetamine or amphetamine derivative can have, are preferably alkyl groups. Alkyl groups with one to ten carbon atoms, in particular, methyl, ethyl, propyl, butyl, pentyl, hexyl, heptyl or octyl groups, are especially preferred.

The alkyl groups can basically be cyclized, linear or branched. However, short-chain (up to 5 carbon atoms) and unbranched alkyl groups, in particular methyl and/or ethyl groups, are especially preferred.

The at least one substituent different from H on the phenyl ring is also preferably an alkyl group. With respect to preferred compositions, reference can be made without restriction to the above account relating to the residues different from H, which the amphetamine or amphetamine derivative can have.

Furthermore, however, preferred compositions are also conceivable in which the at least one substituent different from H is an OH, alkoxy, aryl or aryloxy group. In particular, an amphetamine or amphetamine derivative can, in addition to one or more such substituents, also have one or more alkyl groups.

Purely for the sake of completeness, it should be mentioned that all positions on the phenyl ring that are not occupied by a fluorine or by a substituent different from H, in particular, have an H as substituent, i.e., are unsubstituted.

In an especially preferred compositions, the fluorine-substituted amphetamine or amphetamine derivative has a phenyl ring of Formula (II):

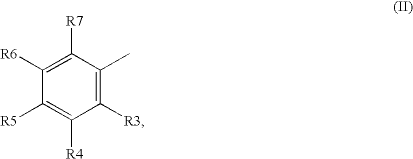

where at least two of the substituents R3 to R7, in particular at least R4 and R5, are fluorine and at least one of the residues R1 or R2 is different from H.

The residue R1 and/or the residue R2 is preferably an ethyl group. Especially preferably, in particular one of the two residues is an ethyl group and the other H.

Moreover, the preferred amphetamine or amphetamine derivative has Formula (III):

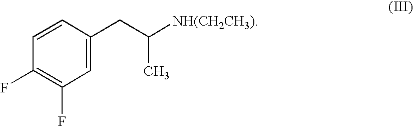

In another especially preferred composition, the amphetamine or amphetamine derivative has a phenyl ring of Formula (II):

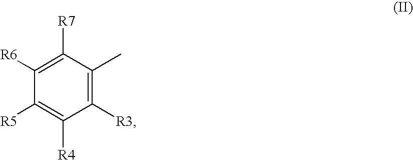

in which at least three of R3 to R7 are fluorine, one of the residues R1 or R2 is H and the other is H or is different from H, in particular, an alkyl group.

In this case, the residues R1 and R2 are preferably equal to H.

Moreover, the amphetamine or amphetamine derivative has, in this further especially preferred composition, Formula (IV):

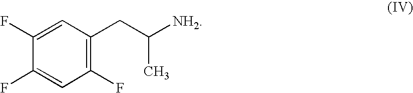

In a third especially preferred composition, the amphetamine or amphetamine derivative has a phenyl ring of Formula (II):

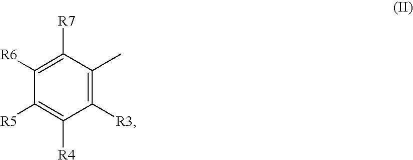

where at least one of the substituents R3 to R7 is fluorine and at least one other is an alkyl group.

In particular, in this compositions, at least R4 is fluorine and at least R5 is an alkyl group or at least R5 is fluorine and at least R4 is an alkyl group.

The at least one alkyl group is preferably a methyl group.

Preferably, in this compositions, one of the residues R1 or R2 is equal to H whereas the other is equal to H or is different from H, in particular, an alkyl group.

Especially preferably, the residues R1 and R2 are equal to H.

Moreover, the amphetamine or amphetamine derivative has, in this third especially preferred composition, the Formulas (V) or (VI):

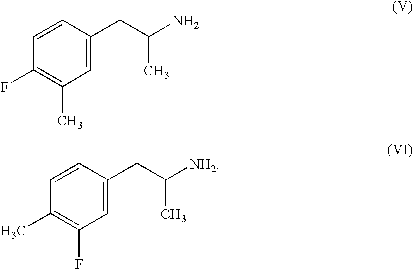

The production of amphetamines or amphetamine derivatives can basically be carried out in various ways. The especially preferred methods of production of the amphetamines or amphetamine derivatives described below are also a subject of this disclosure.

However, production starting from fluorine-substituted benzaldehydes is especially preferred. These can be reacted with nitroethane in the manner of an aldol reaction, followed by reduction of the resultant double bond and of the nitro group. Reduction is preferably carried out by means of a complex hydride, in particular with lithium aluminum hydride. The resultant amino group can then be alkylated by routine methods, for example, by means of methyl formate or acetic anhydride (the intermediates obtained after reaction of the amino compound with these reagents must of course be reduced, for example, with lithium aluminum hydride).

Another especially preferred method of production of amphetamines or amphetamine derivatives starts from fluorine-substituted benzene derivatives, which are reacted with an alanine derivative. Especially suitable alanine derivatives are alanine hydrochloride and phthaloyl alanine. By using suitable Lewis-acid catalysts, the alanine derivative can replace an H on the benzene ring. As a rule, as is already known, there is immediately formation of a carbonyl compound, which can then be reduced. In this way we obtain a fluorine-substituted amphetamine which can then be modified at the nitrogen, if desired.

This procedure can be used especially advantageously for the production of enantiomerically pure amphetamines or amphetamine derivatives. When L-alanine is used, we obtain enantiomerically pure (S)-1-(fluorophenyl)-2-aminopropanes. D-alanine results in the corresponding (R) isomers.

We further provide the use of a fluorine-substituted amphetamine or amphetamine derivative as a medicinal product, in particular, for the treatment of neurological diseases, their sequelae and/or for the treatment of side effects of a therapy of neurological diseases and also, in particular, the use of a fluorine-substituted amphetamine or amphetamine derivative for the production of a medicinal product, in particular, a medicinal product for the treatment of neurological diseases, their sequelae and/or for the treatment of side effects of a therapy of neurological diseases.

Basically any amphetamine or amphetamine derivative that has a phenyl ring, which is substituted with fluorine in at least one position, is suitable for such use.

Especially preferably, an amphetamine or amphetamine derivative is used whose phenyl ring is fluorine-substituted in the at least one position and in at least one other position has a substituent different from H, in particular, an alkyl group.

Preferably the amphetamine or amphetamine derivative that can be used has, on the nitrogen, at least one residue that is different from H, in particular, an alkyl group.

Especially preferably, the amphetamine or amphetamine derivative that can be used is the fluorine-substituted amphetamines or amphetamine derivatives as already described above, in particular, compounds (III), (IV), (V) and (VI), among which once again compound (III) is especially preferred.

In another especially preferred use, the fluorine-substituted amphetamine or amphetamine derivative is a compound according to one of the Formulas (VII) or (VIII):

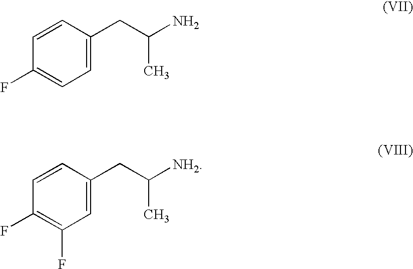

It has already been mentioned that the fluorine-substituted amphetamine or amphetamine derivative is eminently suitable, in particular, for the treatment of neurological diseases and their sequelae.

Moreover, it has been found that the fluorine-substituted amphetamine or amphetamine derivative is, in particular, also suitable for the treatment of side effects of a therapy of neurological diseases.

Preferably the fluorine-substituted amphetamine or amphetamine derivative can, in the therapy of neurological diseases, also be administered for the prevention of side effects.

Therefore, the fluorine-substituted amphetamine or amphetamine derivative surprisingly can also be used in the case of several indications arising simultaneously.

Among the treatable neurological diseases, we should mention, in particular, degenerative diseases of the extrapyramidal motor system, in particular, Parkinson's diseases, especially preferably idiopathic Parkinson's diseases.

The treatable sequelae of the neurological diseases and/or the side effects are, in particular, motor disturbances, multiple system atrophies, dystonic syndromes, dyskinetic syndromes and parkinsonian symptoms, such as tremor.

The therapy is preferably a drug therapy, in particular, with at least one antiparkinsonian active substance which is selected, in particular, from the group comprising dopamine precursors, decarboxylase inhibitors, dopamine agonists, all active substances that act by stimulation of the dopamine receptors, inhibitors of catechol-O-methyltransferase (COMT), inhibitors of monoamine oxidase (MAO) and antagonists of N-methyl-D-aspartate (NMDA) receptors.

In particular, levodopa (L-DOPA) should be mentioned as dopamine precursor. As decarboxylase inhibitors, consideration should be given, in particular, to benserazide or carbidopa. In the case of dopamine agonists, in particular, we may mention bromocriptine, apomorphine, cabergoline, pramipexole, ropinirole, pergolide, dihydro-alpha-ergocryptine or lisuride. As inhibitors of catechol-O-methyltransferase (COMT), consideration may be given, in particular, to entacapone or tolcapone. In particular, selegiline may be mentioned as an example of inhibitors of monoamine oxidase (MAO). As antagonists of N-methyl-D-aspartate (NMDA) receptors, consideration may be given, in particular, to amantadine or budipine.

Side effects of medicinal products with the stated antiparkinsonian active substances are, in particular, all forms of dyskinesias, in particular, chorea-type, dystonic, ballismic and muscle-cramp dyskinesias, and motor (reactive) fluctuations or psychotic states.

Preferably, amphetamines or amphetamine derivatives can also be used for the treatment of so-called “tardive” dyskinesias, which can be induced by neuroleptics.

In particular, the use comprises the treatment of L-DOPA-induced side effects, in particular, of L-DOPA-induced dyskinesias.

Basically, the fluorine-substituted amphetamine and amphetamine derivative are suitable for the treatment of extrapyramidal motor disturbances of all kinds. In particular, we should mention Parkinson syndromes, dyskinetic, chorea-type or dystonic syndromes (in particular, Huntington's chorea), extrapyramidal motor side effects of neuroleptics, tremor, Gilles de la Tourette syndrome, ballismus, muscle cramps, restless leg syndrome or Wilson's disease.

We also provide pharmaceutical compositions. This comprises at least one fluorine-substituted amphetamine or amphetamine derivative as active substance.

The at least one fluorine-substituted amphetamine or amphetamine derivative has already been described within the scope of the description of the use. Reference is hereby expressly made to the relevant statements.

Treatment of a neurological disease with a pharmaceutical composition can completely replace a treatment with a medicinal product containing one of the aforementioned conventional active substances.

In preferred forms, the fluorine-substituted amphetamine or amphetamine derivative is present in a composition as the sole active substance. However, it is also possible to use a mixture of various fluorine-substituted amphetamines or amphetamine derivatives.

Moreover, it is preferable for a pharmaceutical composition to have at least one pharmaceutically compatible carrier. Correspondingly suitable carriers are known by a person skilled in the art.

In another preferred form, the pharmaceutical composition has a combination of the at least one fluorine-substituted amphetamine or amphetamine derivative and at least one conventional active substance, in particular, at least one conventional antiparkinsonian active substance.

For this, consideration may be given, in particular, to at least one active substance from the group comprising dopamine precursors, decarboxylase inhibitors, dopamine agonists, all active substances that act by stimulation of the dopamine receptors, inhibitors of catechol-O-methyltransferase (COMT), inhibitors of monoamine oxidase (MAO) and antagonists of N-methyl-D-aspartate (NMDA) receptors.

Preferred decarboxylase inhibitors, dopamine agonists, inhibitors of catechol-O-methyltransferase, inhibitors of monoamine oxidase (MAO) and antagonists of N-methyl-D-aspartate (NMDA) receptors have already been mentioned. Reference is hereby made to the corresponding details.

Especially preferably, the at least one antiparkinsonian active substance is L-DOPA, which has already been mentioned several times.

Further features and advantages will become clear from the following description of preferred forms based on examples. The individual features can in each case be realized on their own or several can be realized in combination with one another.

### EXAMPLES

(1) Amphetamines or amphetamine derivatives can basically be produced in several ways. Syntheses starting from benzaldehydes, which lead via 1-(fluorophenyl)-2-nitropropenes to 1-(fluorophenyl)-2-aminopropanes, are especially preferred. The latter can optionally be reacted further and, in particular, functionalized on the amino group.

### Step 1: Preparation of 1-(fluorophenyl)-2-nitropropenes

Starting from the benzaldehydes, in a first step 1-(fluorophenyl)-2-nitropropenes are produced. For this, 208 mmol of aldehyde (3,4-difluorobenzaldehyde, 3-fluoro-4-methylbenzaldehyde, 3-methyl-4-fluorobenzaldehyde, 2,5-difluorobenzaldehyde or 2,4,5-trifluorobenzaldehyde), 208 mmol (16.2 g, 15.5 ml) of nitroethane, 44 mmol (10 g, 10.6 ml) of γ-aminopropyltriethoxysilane and 44 mmol (2.6 g, 2.5 ml) of glacial acetic acid in 25 ml of methanol are stirred for several days. Crystals are precipitated, depending on the aldehyde. These are filtered off, washed with cold aqueous methanol (10% water), and dried. Otherwise it is diluted with water and extracted with diethyl ether three times.

The various 1-(fluorophenyl)-2-nitropropenes were isolated at yields between 60% and 90% and characterized by ¹H-NMR.

### Step 2: Preparation of 1-(fluorophenyl)-2-aminopropanes

The 1-(fluorophenyl)-2-nitropropenes produced according to step 1 can be reacted as follows:
Dissolve 0.1 mol of a 1-(fluorophenyl)-2-nitropropene in diethyl ether or tetrahydrofuran and add dropwise to 0.22 mol (8.38 g) of lithium aluminum hydride dissolved in diethyl ether, stir overnight and then hydrolyze with 8 ml of water, 8 ml of 15% sodium hydroxide solution and 24 ml of water. Filter off the precipitated aluminum hydroxide and wash the precipitate with diethyl ether. Dry the combined diethyl ether phases over solid potassium hydroxide and draw off the diethyl ether in vacuum. The oil that remains is taken up in a just sufficient amount of dilute hydrochloric acid or dilute methanesulfonic acid and the resultant weak acid solution is extracted twice with ethyl acetate and twice with diethyl ether. The extracts are discarded. The aqueous solution is concentrated to dryness under vacuum and the residue is reprecipitated from boiling ethyl acetate. The hydrochlorides or the methanesulfonates of 1-(fluorophenyl)-2-aminopropanes are obtained at yields between 75 and 85%.

Data for some of the compounds prepared:
1-(3,4-Difluorophenyl)-2-aminopropane hydrochloride:
¹H-NMR in d₆-DMSO on 400 MHz instrument:
   CH₃: d: 1.14 ppm (³J=6.4 Hz)
   CH₂: dd: 2.73 ppm (²J=13.4, ³J=8.4 Hz), dd: 3.05 ppm (²J=13.4, ³J=5.6 Hz)
   CH: m: 3.40 ppm
   Ph: m (1H): 7.11 ppm; m (2H): 7.36 ppm;
   NH₃: brd (3H): 8.35 ppm
¹⁹F{¹H}-NMR in d₆-DMSO on 400 MHz instrument:
   d: −138.79 ppm (³J=22.5 Hz), d: −141.65 ppm (³J=22.5 Hz)
1-(2,5-Difluorophenyl)-2-aminopropane methanesulfonate:

¹H-NMR in d₆-DMSO on 400 MHz instrument:
CH₃: d: 1.11 ppm (³J=6.5 Hz)
CH₃: s: 2.35 ppm
CH₂: dd: 2.76 ppm (²J=13.6, ³J=8.7 Hz), dd: 2.96 ppm (²J=13.6, ³J=5.5 Hz)
CH: m: 3.44 ppm
Ph: m 1H, 7.17 ppm; m 2H, 7.26 ppm;
NH₃: 7.94 ppm
¹⁹F-NMR in d₆-DMSO on 400 MHz instrument:
   m 1F: −118.67 ppm, m 1F: −123.22 ppm
1-(2,4,5-Trifluorophenyl)-2-aminopropane hydrochloride (corresponds to the compound according to Formula IV):
¹H-NMR in d₆-DMSO on 250 MHz instrument:
   CH₃: d: 1.13 ppm (³J=6.4 Hz)
   CH₂: dd: 2.79 ppm (²J=13.66, ³J=8.32 Hz), dd: 2.99 ppm (²J=13.66, ³J=5.68 Hz)
   CH: m: 3.39 ppm
   Ph: m 2H, 7.53 ppm
   NH₃: brd 3H, 8.33 ppm
1-(3-Fluoro-4-methylphenyl)-2-aminopropane hydrochloride (corresponds to the compound according to Formula VI):
¹H-NMR in d₆-DMSO on 250 MHz instrument:
   CH₃: d: 1.10 ppm (³J=6.4 Hz)
   CH₃: d (not resolved): 2.16 ppm
   CH₂: dd: 2.66 ppm (²J=13.3, ³J=8.7 Hz), dd: 3.05 ppm (²J=13.3, ³J=4.9 Hz)
   CH: m: 3.35 ppm
   Ph: m 2H, 6.99 ppm, m 1H, 7.18 ppm
   NH₃: brd 3H, 8.35 ppm
¹⁹F-NMR in d₆-DMSO on 250 MHz instrument:
   m: −119.74 ppm
1-(3-Methyl-4-fluorophenyl)-2-aminopropane hydrochloride (corresponds to the compound according to Formula V):
¹H-NMR in d₆-DMSO on 250 MHz instrument
   CH₃: d: 1.10 ppm (³J=6.6 Hz)
   CH₃: d: 2.17 ppm (⁴J=1.7 Hz)
   CH₂: dd: 2.62 ppm (²J=13.5, ³J=9.34 Hz), dd: 3.03 ppm (²J=13.5, ³J=5.1 Hz)
   CH: m: 3.32 ppm
   Ph: m: 7.06 ppm;
   NH₃: brd 3H, 8.34 ppm
¹⁹F-NMR in d₆-DMSO on 250 MHz instrument:
   m: −122.88 ppm

### Step 3: Introduction of an N-Methyl Group

A methyl group can be introduced in a subsequent step into 1-(fluorophenyl)-2-aminopropanes prepared according to step 2. In particular, the procedure can be as follows:
Dissolve 0.1 mol of 1-(3,4-difluorophenyl)-2-aminopropane hydrochloride in water. Make the solution basic with sodium hydroxide solution, extract three times with diethyl ether and dry the combined extracts over solid potassium hydroxide. Decant the solution from the drying agent and remove the ether under vacuum. The oil that remains is taken up in methyl formate and is stirred overnight in the autoclave at 20 bar H₂ pressure and 70° C. After removing the pressure, the solution is concentrated by evaporation, taken up in tetrahydrofuran and added dropwise to 0.1 mol (3.8 g) of lithium aluminum hydride in tetrahydrofuran and stirred for 24 h at 45° C. Then it is hydrolyzed with 4 ml of water, 4 ml of 15% sodium hydroxide solution and 12 ml of water. The precipitated aluminum hydroxide is filtered off and the aluminum hydroxide is washed with diethyl ether three times. The solvent is removed from the combined ether phases under vacuum and the residue is taken up in a just sufficient amount of dilute hydrochloric acid. This solution is extracted twice with ethyl acetate, then twice with diethyl ether; the extracts are discarded. The aqueous solution is made basic with sodium hydroxide and extracted three times with diethyl ether. Hydrochloric acid is added to the combined ether extracts while stirring vigorously, until the emulsion shows a neutral reaction. The solvents are removed under vacuum and the dried residue is recrystallized from ethyl acetate. As a rule the yield was approx. 80%.

Data for a compound prepared in this way:
1-(3,4-Difluorophenyl)-2-methylaminopropane hydrochloride:
¹H-NMR in d₆-DMSO on 400 MHz instrument:
   CH₃ d: 1.10 ppm (³J=6.2 Hz)
   CH₃ s: 2.53 ppm
   CH₂ dd: 2.70 ppm (²J=13.5, ³J=10.0 Hz), dd: 3.17 ppm (²J=13.5, ³J=4.2 Hz)
   CH m: 3.37 ppm
   Ph m 1H, 7.13 ppm, m 2H, 7.39 ppm;
   NH₂ brd: 9.20 ppm
¹⁹F{¹H}-NMR in d₆-DMSO on 400 MHz instrument:
   d: −138.75 ppm (³J=22.1 Hz), d: −141.60 ppm (³J=22.1 Hz)

### Step 3 (Alternative): Introduction of an N-Ethyl Group

As an alternative to step 3, an ethyl group can also be introduced in a subsequent step into 1-(fluorophenyl)-2-aminopropanes prepared according to step 2. The procedure can be as follows:
Similarly to step 3, transform 0.1 mol of 1-(3,4-difluorophenyl)-2-aminopropane hydrochloride to the free base. Add 0.1 mol (7.91 g, 8.1°ml) of pyridine to the ethereal solution decanted from the potassium hydroxide. While cooling with ice, now add 0.1 mol (10.2 g, 9.5 ml) of acetic anhydride in portions and stir overnight at room temperature. On completion of reaction, wash three times with 2n hydrochloric acid, once with potassium hydrogencarbonate solution, and three times with water; dry and concentrate by evaporation. The residue is taken up in tetrahydrofuran and, as in step 3, reduced with lithium aluminum hydride and processed. Once again the yield was as a rule approx. 80%.

Data for a compound prepared in this way:
1-(3,4-Difluorophenyl)-2-ethylaminopropane hydrochloride (corresponds to compound III):
¹H-NMR in d₆-DMSO on 400 MHz instrument:
   CH₃ d: 1.12 ppm (³J=6.3 Hz)
   CH₃ t: 1.26 ppm (³J=7.0 Hz)
   CH₂ m: 2.98 ppm
   CH₂ dd: 2.70 ppm (²J=13, ³J=10 Hz), dd: 3.28 ppm (²J=13, ³J=3 Hz)
   CH m: 3.40 ppm
   Ph m 1H, 7.13 ppm, m 2H, 7.39 ppm;
   NH₂ brd: 9.32 ppm
¹⁹F{¹H}-NMR in d₆-DMSO on 400 MHz instrument:
   d: −138.75 ppm (³J=22.4 Hz), d: −141.63 ppm (³J=22.4 Hz)_{,}

(2) Another especially preferred route for production of amphetamines or amphetamine derivatives starts from substituted benzenes. The synthesis of 1-(2,5-difluorophenyl)-2-aminopropane methanesulfonate from 1,4-difluorobenzene and alanine hydrochloride is described below:
Melt 48 mmol (6.0 g) of alanine hydrochloride and 48 mmol (11 g) of antimony(III) chloride at 70° C. under argon and add 48 mmol (10 g) of phosphorus pentachloride. HCl gas is evolved. After 30 minutes, evacuate to remove the phosphorus oxychloride that formed. Add 48 mmol (5.48 g, 5.0 ml) of 1,4-difluorobenzene and 150 mmol (20.0 g) of aluminum chloride to the melt that remains. Then stir for 24 hours at 70° C. Stir the black reaction solution while still hot into a cold (−10° C.) solution of 0.9 mol (50.4 g) of potassium hydroxide and 105 mmol (4.0 g) of sodium borohydride in 200 ml of water. Heat the alkaline solution, which contains suspended antimony, for 30 minutes at 40° C. and then shake in 400 ml of 3n hydrochloric acid (considerable foaming), filter to remove the precipitated antimony and extract three times with diethyl ether. Make the aqueous phase strongly alkaline with potassium hydroxide and extract three times with diethyl ether. Dry the combined diethyl ether phases over potassium hydroxide. Neutralize, with methanesulfonic acid, the ether phase decanted from the drying agent, and filter off the precipitated 1-(2,5-difluorophenyl)-1-hydroxy-2-aminopropane methanesulfonate. This methanesulfonate is hydrogenated overnight in 30 ml of methanesulfonic acid at 90° C. and 1 bar with 2 g of 10% Pd/C as catalyst. For processing, dilute with 200 ml of water, adjust the pH to 3 with sodium hydroxide and extract three times with diethyl ether. Then make the aqueous phase strongly alkaline, extract three times with ether and process the combined ether phases as above to 1-(2,5-difluorophenyl)-2-aminopropane methanesulfonate. Recrystallization from ethyl acetate containing a little ethanol gives 4.7 g (37% of theory).

Data for the 1-(2,5-difluorophenyl)-2-aminopropane methanesulfonate obtained:
¹H-NMR in d₆-DMSO on 400 MHz instrument:
   CH₃ d: 1.11 ppm (³J=6.5 Hz)
   CH₃ s: 2.35 ppm
   CH₂ dd: 2.76 ppm (²J=13.6, ³ J=8.7 Hz), dd: 3.96 ppm (²J=13.6, ³J=5.5 Hz)
   CH m: 3.44 ppm
   Ph m 1H, 7.23 ppm, m 2H, 7.35 ppm;
   NH₂ brd: 7.94 ppm
¹⁹F-NMR in d₆-DMSO on 400 MHz instrument:
   m: −118.7 ppm, m: −123.2 ppm

The use of enantiomerically pure alanine yields an optically active product in this synthesis. Thus, L-alanine yields S-(+)-1-(2,5-difluorophenyl)-2-aminopropane methanesulfonate. Correspondingly, D-alanine yields the (R) isomer.

(3) Another especially preferred synthesis of an amphetamine, starting from a substituted benzene, is given below:

### Production of 1-(3,4-difluorophenyl)-2-aminopropane hydrochloride from 1,2-difluorobenzene and N-phthaloyl alanine

Make a slurry of 50 mmol (10.96 g) of phthaloyl alanine (A. K. Bose, F. Greer, C. C. Price, J. Org. Chem, 1958 (23), 1335-1337, M. S. F. Lie Ken Jie, H. B. Lao, D. W. Y. Leung, Lipids, 1990 (25), 260-264) in 60 ml of CH₂Cl₂ and, while cooling with ice, add 50 mmol (10.41 g) of phosphorus pentachloride. After a few minutes a clear solution is obtained; stir this for 1 hour. Then the solvent and the phosphorus oxychloride that formed are removed completely, under vacuum. Then add 60 mmol (6.85 g, 6.2 ml) of 1,2-difluorobenzene and 52.5 mmol (12 g) of sublimed antimony(III) chloride and heat to 40° C. A slightly yellowish solution is formed. Add 105 mmol (14 g) of aluminum chloride in 2 portions to the hot solution. Then stir for 3 h at 65° C. Generally the mixture crystallizes during this time. Leave to cool to room temperature and add 100 ml of dichloromethane. After everything has dissolved, the solution is poured into 200 ml of cold 4-molar hydrochloric acid. Wash the methylene chloride phase three times with 2-molar hydrochloric acid and twice with water, and stir overnight with a potassium hydrogencarbonate solution. After separating the aqueous phase, wash three times with water, dry over magnesium sulfate and concentrate by evaporation. We obtain 1-(3,4-difluorophenyl)-2-phthaloyl aminopropane as a brownish oil, which contains about 12% of 1-(2,3-difluorophenyl)-2-phthaloyl aminopropane, at almost quantitative yield. By fractional crystallization, first from isopropanol, then twice from methyl-t-butyl ether, we obtain pure 1-(3,4-difluorophenyl)-2-phthaloyl aminopropane at approx. 70% yield. This compound can be cleaved, as described by J. O. Osby, M. G. Martin, B. Ganem, Tetrahedron Letters, 1984 (25), 2093-2096, with NaBH₄ in isopropanol/water and then acetic acid to 1-(3,4-difluorophenyl)-1-hydroxy-2-aminopropane. The methanesulfonate of this compound can, as described in (3), be hydrogenated in methanesulfonic acid at 90° C. and purified as hydrochloride. We obtain 1-(3,4-difluorophenyl)-2-aminopropane hydrochloride at 55% yield relative to the alanine used.

The data for this compound have already been given.

When L-alanine is used, we obtain enantiomerically pure (S)-1-(3,4-difluorophenyl)-2-aminopropane hydrochloride. Correspondingly, D-alanine yields the (R) isomer.

(4) Degeneration of dopaminergic neurons is a causative factor for Parkinson's disease in humans. It leads to dopamine depletion in the basal ganglia of the brain (dopamine hypofunction).

Potentially suitable active substances for treatment of Parkinson's disease can be investigated in an animal model. It is desirable, in such an animal model of Parkinson's disease, to achieve reduced functional dopamine activity by blocking dopamine receptors or by destroying dopaminergic neurons.

Next, rats (Sprague-Dawley rats; Charles-River, Sulzfeld, Germany) were established as the animal model. The rats were kept in constant, reproducible conditions. Sprague-Dawley rats weighing about 220 to 300 g were used in the experiment. The rats were kept in groups. A light cycle of 12:12 hours was maintained (light switched on from 07:00 to 19:00 hours). For two weeks before the experiments, all the rats were kept in the same room. Water was made available ad libitum, standard animal feed was supplied once daily in an amount of 12 g per animal. All experiments were carried out between 09:00 and 17:00 hours and complied with international ethical standards and the German animal protection law. It was approved by the Animal Protection Commission, Tübingen Administrative Board, ZP 5/01.

### (4a) Influence of a Fluorine-Substituted Amphetamine or Amphetamine Derivative on Parkinsonian Symptoms in Sprague-Dawley Rats.

Haloperidol-induced catalepsy is used here as a model for parkinsonism. Haloperidol is a dopamine receptor-blocking drug. The catalepsy induced in this way comprises akinesia and rigor. The haloperidol model can be treated with all known clinically effective antiparkinsonism drugs, as described above.

Dopamine hypofunction and the consequent parkinsonian symptoms were generated as follows.

Control Group:
Haloperidol (Haldol, Janssen®, Germany) was diluted with phosphate buffered saline (PBS; Sigma, Deisenhofen, Germany) to a concentration of 0.5 mg per ml. Twelve rats were each given an intraperitoneal (i.p.) injection of this solution with an absolute amount of haloperidol of 0.5 mg per kilogram of body weight and an injection with PBS.

Test Group:
Another twelve rats were treated identically, but instead of the PBS injection were given a fluorine-substituted amphetamine derivative 1-(3,4-difluorophenyl)-2-(N-ethylamino)propane (Formula III) dissolved in PBS. Quantitative assessment of akinesia and rigor in the catalepsy test according to Schmidt et al. (Werner J. Schmidt, Andreas Mayerhofer, Anja Meyer, Karl-A. Kovar, “Ecstasy counteracts catalepsy in rats, an anti-parkinsonian effect?”, Neuroscience Letters 330 (2002) 251-254) showed that akinesia and rigor were significantly less pronounced in the test group than in the control group.

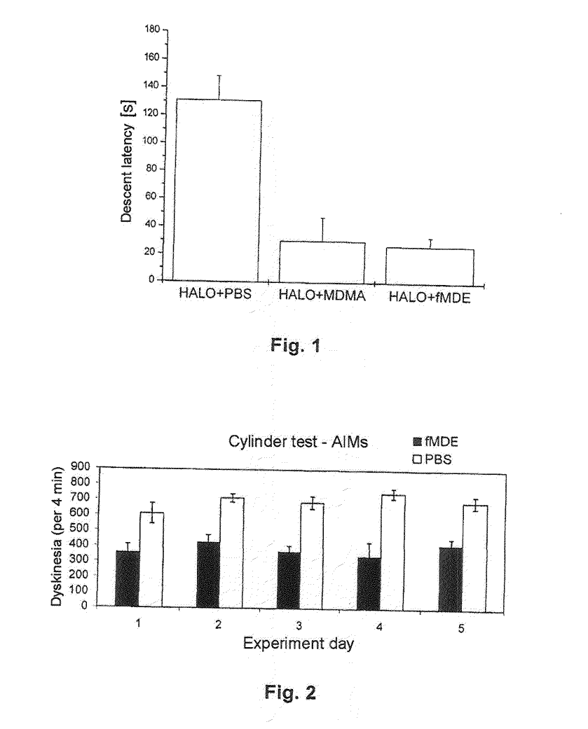

The results of the descent tests are illustrated in FIG. 1. It was found that catalepsy was pronounced in rats after treatment with haloperidol (HALO, 0.5 mg/kg), followed by injection of PBS (vehicle control), with a descent latency of about 130 seconds. The descent latency of rats treated with MDMA (5 mg/kg) was much less, at about 30 seconds, but for the rats treated with 1-(3,4-difluorophenyl)-2-(N-ethylamino)propane (fMDE, Formula (III), 5 mg/kg) it was even lower.

Similar results were achieved with compounds according to Formulas IV, V, VI, VII or VIII.

### (4b) Effect of a Fluorine-Substituted Amphetamine or Amphetamine Derivative on Dyskinesia.

24 rats were put in deep narcosis. One hemisphere of the rats was treated in each case with the neurotoxin 6-hydroxydopamine, which destroyed dopaminergic neurons in this hemisphere. On the side of the body contralateral to the damaged hemisphere, the animals so treated displayed parkinsonian symptoms (hemiparkinsonism). Then the animals were treated intraperitoneally with an antiparkinsonian drug twice daily for 25 days. This comprised L-DOPA-methyl ester in an amount of 10 mg per kg of body weight with benserazide in an amount of 7.5 mg per kg of body weight (Sigma, Deisenhofen, Germany). Under this treatment the rats developed dyskinesias of the contralateral forepaws.

Control Group:
Continuing this treatment, 12 of the 24 rats were treated additionally with PBS.

Test Group:
The other twelve rats were treated additionally with a fluorinated amphetamine derivative 1-(3,4-difluorophenyl)-2-(N-ethylamino)propane (Formula III).

A quantitative assessment showed significantly reduced expression of the dyskinesias in the test group.

The results for the treatment are illustrated in FIG. 2. The dyskinesia was quantified (AIM=abnormal involuntary movements) in the control group on five successive days. The hemiparkinsonoid rats chronically pretreated with L-DOPA were, after treatment with the antiparkinsonian agent (L-DOPA-methyl ester with benserazide (10+7.5 mg/kg)), injected with PBS as vehicle control. For comparison, the dyskinesia quantification of the test group, i.e., the animals additionally treated with 1-(3,4-difluorophenyl)-2-(N-ethylamino)propane (Formula (III), fMDE, 5 mg/kg), was observed. The measurements were in each case recorded 60 minutes after the animals were treated. It was found that the number of AIMs of the animals per 4 min was almost halved by the treatment with fMDE. The effect was already seen on the first day of administration of fMDE. The animals were sacrificed on completion of the experiment.

In addition, the addiction potential of the amphetamines and amphetamine derivatives. In so-called “conditioned place preference” experiments, the addiction potential with a daily dose of 5 mg per kg of body weight was investigated on Sprague-Dawley rats. Such tests, which are known by a person skilled in the art, are for example required as standard by the U.S. Food and Drug Administration (FDA), for analgesics. In studies to date, the animals have shown no signs of addictive behavior.

Similar results were achieved with compounds according to Formulas IV, V, VI, VII or VIII.

1-32. (canceled)

33. A fluorine-substituted amphetamine or amphetamine derivative with the formula (I): 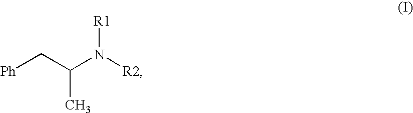
where
a) at least one of the residues R1 or R2 is different from H and Ph is a phenyl ring, which is substituted with fluorine in at least one position
or
b) the residues R1 and R2 independently of one another are H or are different from H and Ph is a phenyl ring, which is substituted with fluorine in at least three positions
or
c) the residues R1 and R2 independently of one another are H or are different from H and Ph is a phenyl ring, which is substituted with fluorine in at least one position and has a substituent different from H in at least one other position.

34. The amphetamine or amphetamine derivative as claimed in claim 33, wherein the residues different from H are alkyl groups.

35. The amphetamine or amphetamine derivative as claimed in claim 33, wherein the residues different from H are alkyl groups with one to ten carbon atoms.

36. The amphetamine or amphetamine derivative as claimed in claim 33, wherein the at least one substituent different from H on the phenyl ring is an alkyl group.

37. The amphetamine or amphetamine derivative as claimed in claim 33, wherein the at least one substituent different from H on the phenyl ring is an alkyl group with one to ten carbon atoms.

38. The amphetamine or amphetamine derivative as claimed in claim 33, wherein the phenyl ring has the formula (II): 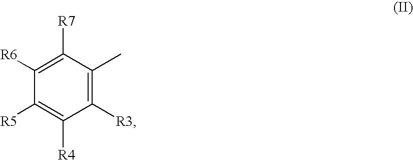
where at least two of the substituents R3 to R7 are fluorine and at least one of the residues R1 or R2 is different from H.

39. The amphetamine or amphetamine derivative as claimed in claim 33, wherein the residue R1 and/or the residue R2 is an ethyl group.

40. The amphetamine or amphetamine derivative as claimed in claim 33, having the formula (III):

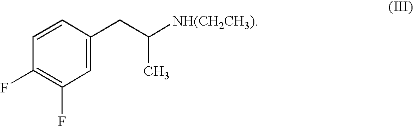

41. The amphetamine or amphetamine derivative as claimed in claim 33, wherein the phenyl ring has the formula (II): 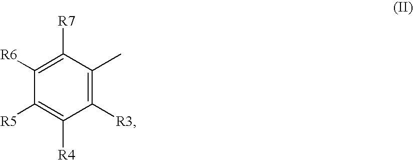
where at least three of R3 to R7 are fluorine, one of the residues R1 or R2 is equal to H and the other is equal to H or is different from H.

42. The amphetamine or amphetamine derivative as claimed in claim 33, wherein the residues R1 and R2 are equal to H.

43. The amphetamine or amphetamine derivative as claimed in claim 42, wherein the amphetamine or amphetamine derivative has the formula (IV):

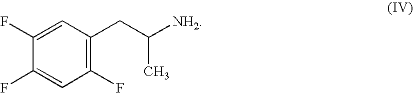

44. The amphetamine or amphetamine derivative as claimed in claim 33, wherein the phenyl ring has the formula (II): 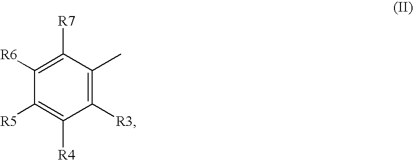
where at least one of the substituents R3 to R7 is fluorine and at least one other is an alkyl group.

45. The amphetamine or amphetamine derivative as claimed in claim 44, wherein at least R4 is fluorine and at least R5 is an alkyl group or at least R5 is fluorine and at least R4 is an alkyl group.

46. The amphetamine or amphetamine derivative as claimed in claim 44, wherein the at least one alkyl group is a methyl group.

47. The amphetamine or amphetamine derivative as claimed in claim 44, wherein one of the residues R1 or R2 is equal to H and the other is equal to H or is different from H.

48. The amphetamine or amphetamine derivative as claimed in claim 44, wherein the residues R1 and R2 are equal to H.

49. The amphetamine or amphetamine derivative as claimed in claim 44, having the formula (V) or (VI):

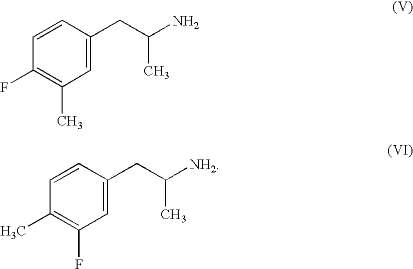

50. A method for treating neurological diseases, their sequelae and/or treating side effects of a therapy of neurological diseases comprising administering a therapeutically effective amount of a fluorine-substituted amphetamine or amphetamine derivative to a patient.

51. The method as claimed in claim 50, wherein the amphetamine or amphetamine derivative has a phenyl ring which is substituted with fluorine in at least one position.

52. The method as claimed in claim 50, wherein the amphetamine or amphetamine derivative has a phenyl ring which is substituted with fluorine in at least one position and in at least one other position has a substituent different from H.

53. The method as claimed in claim 50, wherein the amphetamine or amphetamine derivative has, on the nitrogen, at least one residue different from H.

54. The method as claimed in claim 50, wherein the fluorine-substituted amphetamine or amphetamine derivative has the structure of formula (I): 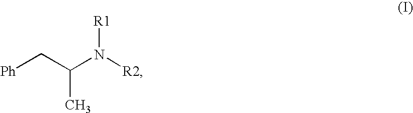
where
a) at least one of the residues R1 or R2 is different from H and Ph is a phenyl ring, which is substituted with fluorine in at least one position
or
b) the residues R1 and R2 independently of one another are H or are different from H and Ph is a phenyl ring, which is substituted with fluorine in at least three positions
or
c) the residues R1 and R2 independently of one another are H or are different from H and Ph is a phenyl ring, which is substituted with fluorine in at least one position and has a substituent different from H in at least one other position.

55. The method as claimed in claim 50, wherein the fluorine-substituted amphetamine or amphetamine derivative is a compound according to one of the formulas (VII) or (VIII):

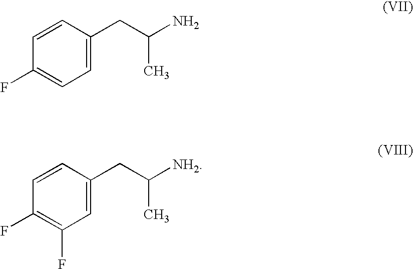

56. The method as claimed on claim 50, wherein the neurological diseases are Parkinson's diseases.

57. The method as claimed in claim 50, wherein the neurological diseases are idiopathic Parkinson's diseases.

58. The method as claim in claim 50, wherein the sequelae and/or side effects are motor disturbances.

59. The method as claimed in claim 50, wherein the therapy is a drug therapy.

60. The method as claimed in claim 50, wherein the therapy is a drug therapy with at least one antiparkinsonian active substance.

61. The method as claimed in claim 60, wherein the at least one antiparkinsonian active substance is selected from the group comprising dopamine precursors, decarboxylase inhibitors, dopamine agonists, all active substances that act by stimulation of the dopamine receptors, inhibitors of catechol-O-methyltransferase (COMT), inhibitors of monoamine oxidase (MAO), antagonists of acetylcholine or N-methyl-D-aspartate (NMDA) receptors and combinations thereof.

62. The method as claimed in claim 50, wherein the sequelae and/or side effects are parkinsonian symptoms.

63. The method as claimed in claim 50, wherein the sequelae and/or side effects are multiple system atrophies and/or dystonic syndromes and/or dyskinetic syndromes and/or tremor.

64. The method as claimed in claim 61, wherein the at least one antiparkinsonian active substance is L-DOPA.

65. The method as claimed in claim 50, wherein the side effects are L-DOPA-induced side effects.

66. The method as claimed in claim 50, wherein the side effects are L-DOPA-induced dyskinesia.

67. A pharmaceutical composition comprising at least one fluorine-substituted amphetamine or amphetamine derivative as active substance.

68. The composition as claimed in claim 67, further comprising at least one pharmaceutically compatible carrier.

69. The composition as claimed in claim 67, further comprising at least one antiparkinsonian active substance.

70. The composition as claimed in claim 69, wherein the at least one antiparkinsonian active substance is L-DOPA.
